(19) United States
(12) Patent Application Publication
(10) Pub. No.: US 2025/0072993 A1
(43) Pub. Date: Mar. 6, 2025

(54) Head-Mounted Display System, Surgical Microscope System and corresponding Method and Computer Program

(71) Applicant: Leica Instruments (Singapore) Pte. Ltd., Singapore (SG)

(72) Inventors: Hakim EL HADDOUCHI, Balgach (CH); Manon ROSTYKUS, Vulliens (CH)

(21) Appl. No.: 18/549,606

(22) PCT Filed: Feb. 24, 2022
(86) PCT No.: PCT/EP2022/054700

§ 371 Date: Sep. 8, 2023

(30) Foreign Application Priority Data
Mar. 8, 2021 (DE) 102021105457.5

(51) Int. Cl. A61B 90/00; A61B 90/20; A61B 90/50; G06F 3/01; G06F 3/16; H04N 5/775; H04N 7/18

(52) U.S. Cl. CPC A61B 90/361; A61B 90/20; H04N 5/775; H04N 7/183; A61B 2090/371; A61B 2090/372; A61B 2090/373; A61B 2090/502; G06F 3/013; G06F 3/167

(57) ABSTRACT

Examples relate to a head-mounted display system, to a surgical microscope comprising a head-mounted display system, and to a corresponding method and computer program. The head-mounted display system comprises one or more displays, one or more storage devices and a control system. The control system is configured to receive an image stream from an external device. The control system is configured to record the image stream using the one or more storage devices upon request of a user of the head-mounted display system. The control system is configured to generate a display signal based on the image stream. The control system is configured to provide the display signal to the one or more displays.

### TECHNICAL FIELD

Examples relate to a head-mounted display system, to a surgical microscope comprising a head-mounted display system, and to a corresponding method and computer program.

### BACKGROUND

Surgical microscope systems are often equipped with microscopes with integrated optical imaging sensors, which are used to record the surgical site. The recorded image can then be displayed on a large screen or in digital oculars, e.g. with an overlay that provides the surgeon with additional context on the surgical site. For example, an overlay of an image-guided system may be superimposed, or a pseudo-color representation of fluorescence emissions of a fluorescent dye being injected in some features of the surgical site.

A major focus in the effort for improving the quality of surgical procedures is the recording of the surgical procedures by a camera, which may be integrated into the surgical microscope. In some countries, such as Singapore, such video recording is mandatory for all surgeries.

### SUMMARY

There may be a desire for an improved concept for recording of a surgery.

This desire is addressed by the subject-matter of the independent claims.

Embodiments of the present disclosure are based on the finding, that the images recorded by a surgical microscope can also be viewed using a head-mounted display, e.g. using virtual-reality goggles or augmented-reality goggles. Such head-mounted displays may be used by a surgeon to control the field of view the surgeon sees through the microscope, e.g. by tracking the movement of the head of the surgeon. Consequently, the images seen by the surgeon are highly specific to the field of view the surgeon chooses. Embodiments may be used to record the images recorded by a surgical microscope, as seen by the surgeon, using a storage device that is integrated within the head-mounted display system itself. As both the recorded images and the resulting field of view of the surgeon is “known” at the head-mounted device, no further exchange between the head-mounted display system and the surgical microscope system is necessary. Furthermore, additional recordings may be performed from the perspective of the surgeon, e.g. audio recordings of voices during surgery, or a view of an outward-facing camera that is part of the head-mounted display, creating a more comprehensive recording of the surgical procedure. The same concept may be used in other contexts as well, e.g. to record the view of an operator of a robot being tasked with clearing an obstruction from a pipe, or to record the view of a bomb-defusal expert that uses a robot to approach a potential explosive device.

Various aspects of the present disclosure relate to a head-mounted display system. The head-mounted display system comprises one or more displays. The head-mounted display system comprises one or more storage devices. The head-mounted display system comprises a control system comprising one or more interfaces and one or more processors. The control system is configured to receive an image stream from an external device. The control system is configured to record the image stream using the one or more storage devices upon request of a user of the head-mounted display system. The control system is configured to generate a display signal based on the image stream. The control system is configured to provide the display signal to the one or more displays. By recording the image stream locally at the head-mounted display system, a foundation is created for generating a comprehensive recording of the sensory experience of the user of the head-mounted display system. Furthermore, the recording may be replayed during usage of the head-mounted display system, e.g. to compare and contrast the current view with the recording.

For example, the control system may be configured to generate the display signal alternately based on a currently received version of the image stream or based on a recorded version of the image stream. In other words, either a live version of the image stream may be shown, or a recorded version may be played back.

In some examples, the control system is configured to extract a still image from the image stream upon request of the user of the head-mounted display system, and to record the still image using the one or more storage devices. Still images may be valuable in documenting the work of the user of the head-mounted device, e.g. at crucial points of a surgery, and may be replayed by the user to compare and contrast the current view with the previously recorded still image.

In addition to the image stream seen by the user, the auditory environment of the user may be recorded. For example, the head-mounted display system may comprise at least one microphone. The control system may be configured to receive an audio signal from the at least one microphone. The control system may be configured to record the audio signal using the one or more storage devices upon request of the user of the head-mounted display system. For example, the audio signal may be recorded to preserve a conversation that occurred during usage of the head-mounted display system, e.g. during surgery.

For example, the head-mounted display system may comprise at least one speaker. The control system may be configured to output the recorded audio signal via the at least one speaker upon request of the user of head-mounted display system. For example, the user of the head-mounted display signal may playback the recorded sound, e.g. to recall a progress of the operation the head-mounted display system is used in.

In some cases, the head-mounted display system may also comprise an outward-facing camera, e.g. to record the environment of the user. In other words, the head-mounted display system may comprise at least one camera. The control system may be configured to receive a further image stream from the at least one camera. The control system may be configured to record the further image stream using the one or more storage devices upon request of a user of the head-mounted display system. For example, the further image stream may be preserved along with the recording of the image stream, to build a more comprehensive recording of the environment.

In case a camera is used, the further image stream may be shown in addition to, or alternatively to, the image stream received from the external device, either in real-time or a recording there of. For example, the control system may be configured to generate the display signal alternately based on the image stream or based on the further image stream.

There are various approaches for controlling the head-mounted display system. For example, the head-mounted display system may be controlled via a haptic control element that is arranged at the head-mounted display system. For example, the head-mounted display system may comprise an electro-mechanical control element that is coupled to the control system.

The control system may be configured to obtain a control signal from the electro-mechanical control element. The control system may be configured to control the recording of the image stream and/or a playback of the recorded image stream based on the control signal. For example, the electro-mechanical control element may be a rotary button, configured to generate the control signal based on a rotation and actuation of the rotary button. An electro-mechanical control element, such as a rotary button, may provide an intuitive way of controlling the head-mounted display system.

As introduced above, the head-mounted display system may comprise at least one microphone, and the control system may be configured to receive an audio signal from the at least one microphone. The control system may be configured to perform speech recognition on the audio signal, and to control the recording of the image stream and/or a playback of the recorded image stream based on the speech recognition. Speech recognition may enable a control of the head-mounted display system without manual interaction with the head-mounted display system, e.g. in order to preserve a sterile environment.

Alternatively or additionally, a gaze-detection system may be used to control the head-mounted display system. The head-mounted display system may comprise a gaze-detection system, configured to determine a direction of a gaze of the user of the head-mounted display system. The control system may be configured to control the recording of the image stream and/or a playback of the recorded image stream based on the direction of the gaze of the user. Gaze detection may enable a control of the head-mounted display system without manual interaction with the head-mounted display system, e.g. in order to preserve a sterile environment.

In various examples, the control system is configured to generate the display signal with a user interface with a user interface element related to the control of the recording of the image stream and/or the playback of the recorded image stream. For example, the control system may be configured to control the recording of the image stream and/or a playback of the recorded image stream based on the direction of the gaze of the user if the direction of the gaze of the user is directed towards the user interface element. For example, the user may gaze at a virtual button being shown in the user interface, and actuate the button by gazing at the virtual button.

In general, various types of memory or storage may be used to save the various recordings. For example, the one or more storage devices may comprise local memory and/or an interface for a removable storage device.

As is evident from the above description, the head-mounted display system may comprise a wide range of different components. A holding structure, such as a frame or case of VR/AR goggles, may be used to contain the various components. For example, the head-mounted display system may be a pair of virtual reality goggles. For example, the head-mounted display system may comprise a holding structure that is mechanically coupled to the one or more displays, the one or more storage devices and the control system.

As pointed out above, the proposed concept may be used with a surgical microscope system. Accordingly, the control system may be configured to receive the image stream from a microscope of a surgical microscope system. Various aspects of the present disclosure further relate to a surgical microscope system comprising a microscope and the head-mounted display system introduced above.

Various aspects of the present disclosure relate to a corresponding method for a head-mounted display system. The method is performed by the head-mounted display system. The method comprises receiving an image stream from a device that is external to the head-mounted display system. The method comprises recording the image stream using one or more storage devices of the head-mounted display system upon request of a user of the head-mounted display system. The method comprises generating a display signal based on the image stream. The method comprises providing the display signal to one or more displays of the head-mounted display system.

Various aspects of the present disclosure relate to a corresponding computer program with a program code for performing the above method when the computer program is executed on a processor.

### SHORT DESCRIPTION OF THE FIGURES

Some examples of apparatuses and/or methods will be described in the following by way of example only, and with reference to the accompanying figures, in which

FIG. 1a shows a schematic diagram of an example of a head-mounted display system;

FIG. 1b shows a block diagram of an example of a control system for a head-mounted display system;

FIG. 2 shows a schematic diagram of an example of a surgical microscope system;

FIG. 3 shows a flow chart of an example of a method for a head-mounted display system;

FIG. 4 shows a schematic diagram of an example of a head-mounted display system;

FIG. 5 shows a schematic diagram of an example of a head-mounted display system;

FIG. 6 shows a flow chart of a first example related to video recording;

FIG. 7 shows a flow chart of a second example related to video recording;

FIG. 8 shows a flow chart of a third example related to video recording;

FIG. 9 shows a flow chart of a fourth example related to video recording;

FIG. 10 shows a flow chart of a first example related to video replaying;

FIG. 11 shows a flow chart of a second example related to video replaying;

FIG. 12 shows a flow chart of a first example related to voice recording;

FIG. 13 shows a flow chart of a second example related to voice recording;

FIG. 14 shows a flow chart of a third example related to voice recording; and

FIG. 15 shows a schematic diagram of a system.

### DETAILED DESCRIPTION

Various examples will now be described more fully with reference to the accompanying drawings in which some examples are illustrated. In the figures, the thicknesses of lines, layers and/or regions may be exaggerated for clarity.

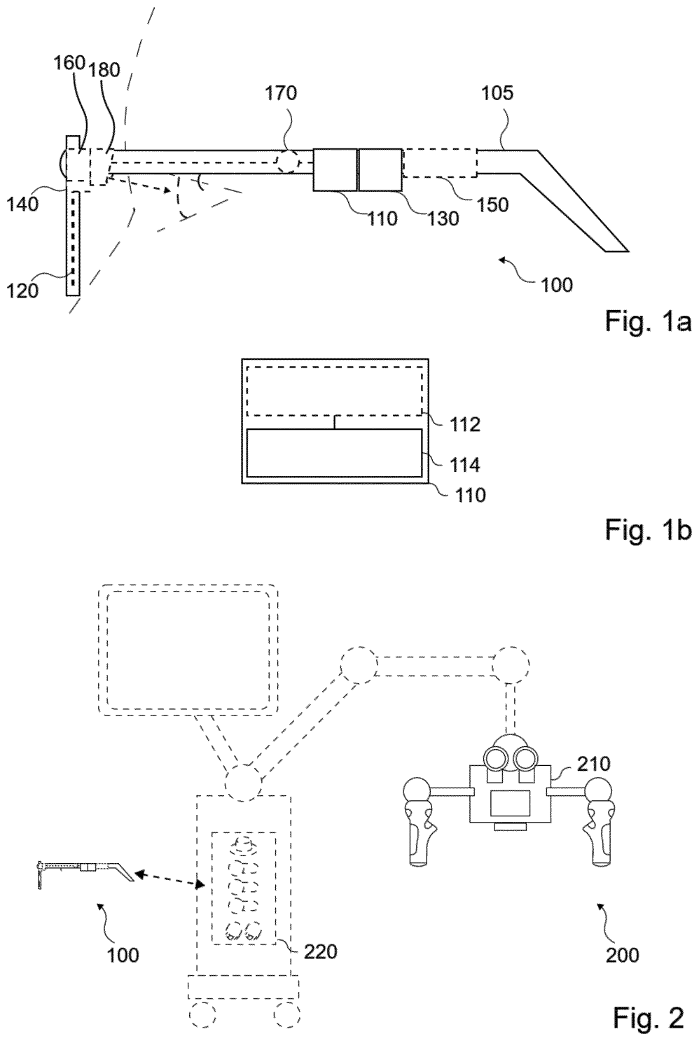

FIG. 1a shows a schematic diagram of an example of a head-mounted display system. The head-mounted display system 100 comprises one or more displays 120, one or more storage devices 130 and a control system 110. The control system 110 is shown in more detail in FIG. 1b FIG. 1b shows a block diagram of an example of a control system 110 for a head-mounted display system. The control system 110 comprises one or more interfaces 112 and one or more processors 114. In general, the one or more processors 110 may be configured to provide the functionality of the control system, e.g. in conjunction with the one or more interfaces 112 (for exchanging information with external entities and/or with other components of the head-mounted display system). Optionally, the control system may comprise one or more storage devices, e.g. the one or more storage devices 130 or one or more storage devices that are separate from the one or more storage devices of the head-mounted display system 100.

The control system is configured to receive an image stream from an external device 210 (e.g. via the one or more interfaces 112). The control system is configured to record the image stream using the one or more storage devices 130 upon request of a user of the head-mounted display system. The control system is configured to generate a display signal based on the image stream. The control system is configured to provide the display signal to the one or more displays.

Various embodiments of the present disclosure relate to a head-mounted display system and/or to a corresponding control system, method and computer program for a head-mounted displays system. In general, a head-mounted display system is, as the name indicates, characterized by two features—it comprises one or more displays 120, and it is head-mounted, i.e. to be worn by the user on, at, or with the head. For example, the head-mounted display system may be worn by a user. For example, the head-mounted display system may be a pair of virtual reality goggles or a pair of augmented-reality glasses. Consequently, the head-mounted display system may be held or top of the nose, and optionally ears of the user, and may be, optionally, held by an elastic strap around the back of the head. The head-mounted display system comprises one or more displays 120, which are in the main field of view of the user of the head-mounted displays system. For example, in case the head-mounted displays system is a pair of virtual reality goggles, one larger display or two smaller displays may be arranged in front of the eyes of the user, i.e. arranged in the field of view of the user. Optionally, the pair of reality goggles may comprise a shield (e.g. blinds) to shield the eyes of the user illumination from the environment outside the pair of virtual goggles. In case the head-mounted display system is a pair of augmented-reality glasses, two smaller semi-transparent displays may be arranged in front of the eyes of the user, so the user can see, at the same time, what is displayed by the one or more displays and the environment around the user. For example, the one or more displays 120 may be one or more Liquid Crystal Displays (LCDs) or one or more Organic Light-Emitting Diode (OLED)-based displays. Alternatively, the one or more displays 120 may be implemented using one or more projectors and one or more projection surfaces. For example, the “glasses” of the augmented-reality glasses may be used as projection surfaces.

The individual components of the head-mounted display system may be held (together) by a holding structure 105, such as a case of the virtual reality goggles or frames of the augmented reality glasses. In other words, the head-mounted display system may comprise a holding structure 105 that is mechanically coupled to the one or more displays, the one or more storage devices and the control system. In other words, the holding structure 105 may hold, and/or contain, the one or more displays, the one or more storage devices and the control system. Furthermore, the holding structure 150 may hold, contain and/or be mechanically coupled to one or more optional components of the head-mounted display system, such as at least one of one or more microphones 140, one or more speakers 150, an (outward-facing) camera 160, an electro-mechanical input device 170 (such as a rotary button), a head-tracking system 180, and a gyroscope.

The head-mounted display system is controlled by the control system. For example, the control system may be used for providing a user interface of the head-mounted display system, for obtaining input signals and providing output signals, and/or for performing data processing in the head-mounted display system. For example, the control system may be integrated within the head-mounted display system, such that the data processing used in the head-mounted display system is performed on-device. For example, the head-mounted display system may be operated (at least partially) independently from an external control system.

In general, the control system is used for three things-receiving input data, such as the image stream, storing the input data using the one or more storage devices upon request of the user, and playing back the received or stored input data. For example, such a system may be used in a surgical environment, to receive a live image stream received from microscope, store the live image stream upon request (e.g. to perform quality control after the surgical procedure, or to enable the surgeon or assistant to recall what happened earlier during the surgical procedure), and to display either the live image stream from the microscope or the image stream that has been stored upon request of the surgeon. Thus, the control system may be used for receiving, processing and playing back the image stream (from the microscope). In the following, the examples given relate to the use of the head-mounted display system with a surgical microscope system (e.g. as shown in FIG. 2). However, the proposed concept is not limited to surgical settings. For example, the same concept may be used in other contexts as well, e.g. to record and playback the view of an operator of a robot being tasked with clearing an obstruction from a pipe, or to record and replay the view of a bomb-defusal expert that uses a robot to approach a potential explosive device.

The control system is configured to receive the image stream from an external device 210. For example, the image stream may be a stream of image frames, such as video stream. For example, the image stream may comprise a sequence of image frames that are received at, and/or displayed at, a pre-defined frame rate (e.g. 24, 30 or 60 frames per second). As pointed out above, the image stream may originate from a microscope of a surgical microscope system. In other words, the control system may be configured to receive the image stream from a microscope 210 of a surgical microscope system 200 (shown in FIG. 2). Alternatively, the control system may be configured to receive the image stream from a different device, such as an endoscope, a robot being used for clearing tubes or a bomb-defusal robot. For example, the image stream may be received via a wire of wirelessly. For example, one of the one or more interfaces may be a wireline interface for receiving the image stream, or one of the one or more interfaces may be a wireless interface, such as a Bluetooth interface or a WiFi interface (i.e. an interface for communicating according to one of the variants of standard IEEE Institute of Electrical and Electronics Engineers 802.11).

The control system is configured to record the image stream using the one or more storage devices upon request of the user, e.g. the surgeon, of the head-mounted display system. For example, the control system may be configured to start the recording in response to a first command of the user, and subsequently terminate the recording in response to a second command of the user. In some examples, the image stream may be continuously stored within the one or more storage devices once the recording is started. In some other examples, the image stream may be recorded within a volatile memory of the control system, and stored within the one or more storage devices once the recording is terminated (or once an insufficient amount of volatile memory is available). For example, the one or more storage devices may comprise local memory, e.g. local flash-based memory. Alternatively or additionally, the one or more storage devices may comprise an interface for a removable storage device, such as a memory card. For example, the one or more storage devices may comprise an interface, e.g. a card slot, for a removable storage device, such as one of a Secure Digital (SD) card, micro-SD card, a memory stick, and a compact flash (express) card. In some examples, the one or more storage devices may also comprise the removable storage device.

In addition to the image stream, single images may be recorded and stored separately upon request of the user, e.g. to have a still-image recording of a particularly relevant view of the suer. For example, the control system may be configured to extract a still image from the image stream (or from a further image stream as introduced in the following) upon request of the user of the head-mounted display system, and to record the still image using the one or more storage devices. For example, the still image may be recorded separately from the image stream. In some cases, the still image may be recorded at a higher quality than the single frames of the image stream.

Furthermore, an environment of the user (and thus of the head-mounted display system) may be recorded, e.g. to establish a comprehensive region of the things seen by the user and of the things that happen around (i.e. in the environment of) the user. For example, sound spoken by the user or occurring around the user (e.g. spoken by other surgical staff) may be recorded in addition of the image stream. Consequently, the head-mounted display system may comprise at least one microphone 140 (which may also be used to control the head-mounted display system). The control system may be configured to receive an audio signal from the at least one microphone. For example, the audio signal may be received as an analog signal by the control system, and digitized by the control system. Alternatively, the audio signal may be received as a digital signal. In this case, the audio signal may be digitized by the microphone or by a separate analog-to-digital converter. The control system may be configured to record the audio signal using the one or more storage devices upon request of the user of the head-mounted display system. For example, the audio signal may be recorded with the image stream (e.g. as audio track of a video comprising the image stream), or separately from the image stream. Additionally or alternatively, the environment of the user may be recorded by at least one camera, which may be part of the head-mounted display system, or which may be external to the head-mounted display system, e.g. integrated within a surgical theatre. In other words, the head-mounted display system may comprise at least one camera 160. For example, the at least one camera may be an outward-facing camera, e.g. a camera for recording the environment of the user of the head-mounted display system. The at least one camera may record the field of view of the user (i.e. the field of view without taking account obstructions by the one or more displays). The control system may be configured to receive a further image stream from the at least one camera, and to record the further image stream using the one or more storage devices upon request of a user of the head-mounted display system. For example, the further image stream may be recorded similar to the image stream. For example, the further image stream may be stored together with the image stream (e.g. as second video track in a common video file), or separate from the image stream.

The control system is further configured to generate the display signal based on the image stream, and, if the optional further image stream is obtained, alternately based on the image stream or based on the further image stream. In this context, the term “generate based on the image stream/further image stream” indicates, that the display signal comprises at least a portion of the image stream. However, the display signal is not necessarily generated based on the “live” image stream or further image stream, but may be generated either based on the “live” image stream or “live” further image stream or based on a recorded and stored version of the image stream or further image stream. In other words, the control system may be configured to generate the display signal alternately based on a currently received version of the image stream or further image stream or based on a recorded version of the image stream or further image stream. In some cases, the display signal may also be generated based on a still image that has been recorded (the still image being extracted from the image stream or further image stream). For example, the display signal may comprise a currently received (i.e. “live”) version of the image stream or further image stream, a recorded version of the image stream or further image stream, or a recorded still image that is extracted from the image stream or the further imager stream. In various examples, the display signal may further comprise a graphical user interface. In other words, the control system may be configured to generate a user interface, and to generate the display signal with the user interface.

The control system is configured to provide the display signal to the one or more displays 120. In general, the display signal may be a signal for driving (e.g. controlling) the one or more displays 120. For example, the display signal may comprise video data and/or control instructions for driving the one or more displays. For example, the display signal may be provided via one of the one or more interfaces 112 of the control system. Accordingly, the control system 110 may comprise a video interface 112 that is suitable for providing the video signal to the one or more displays.

Additionally, the audio recording may be provided to the user via at least one speaker 150 of the head-mounted display system. Consequently, the head-mounted display system may comprise the at least one speaker 150. The control system may be configured to output the recorded audio signal via the at least one speaker upon request of the user of head-mounted display system. In other words, the recorded audio signal may be played back, upon request of the user, via the at least one speaker.

As mentioned above, both the recording and the playback of the image stream and/or audio signal are triggered upon request of the user. In general, the request of the user may be received via an input device of the head-mounted display system. For example, the control system may be configured to obtain one or more requests of the user, e.g. from the user, via an input device. In general, there are a wide variety of suitable input devices.

For example, the input device may be a haptic input device, such as one or more buttons or other haptic input devices that, if triggered, provide a signal to the control system. Thus, such input devices may also be denoted “electro-mechanical input devices” or “electro-mechanical control elements”, as they generate an electrical signal based on a mechanical actuation of the respective input device. For example, the head-mounted display system may comprise an electro-mechanical control element 170 that is coupled to the control system. The control system may be configured to obtain a control signal from the electro-mechanical control element, and to control the recording of the image stream and/or a playback of the recorded image stream based on the control signal. For example, the control signal may represent an actuation of the electro-mechanical control element by the user of the head-mounted display system. For example, when the electro-mechanical control element is actuated, e.g. if a button is pushed, the control signal may represent the actuation of the electro-mechanical control element.

A particular type of electro-mechanical control element that is suitable for controlling the user interface is a rotary button, i.e. a button that can be rotated and pushed. In other words, the electro-mechanical control element may be a rotary button, configured to generate the control signal based on a rotation and (push) actuation of the rotary button. For example, the rotary button may be actuated by rotating the rotary button or by pushing the rotary button. For example, the rotation may be used for selection of a menu item of the user interface, and the push-based actuation may be used to confirm the selection. For example, the control system may be configured to control the recording of the image stream, further image stream (including the still image) or audio signal and/or playback of the image stream, further image stream or audio signal based on the control signal provided by the electro-mechanical control element, e.g. the rotary button.

Alternatively, the user interface may be controlled via voice control. For example, the audio signal might not (only) be recorded, but (additionally) used to control the user interface. For example, the control system may be configured to perform speech recognition on the audio signal. For example, the control system may be configured to identify keywords or key phrases, such as “start recording (image stream/camera image stream/audio)”, “stop recording (image stream/camera image stream/audio)”, “playback (image stream/camera image stream/audio): etc. within the audio signal. The control system may be configured to control the recording of the image stream, further image stream (including the still image) or audio signal and/or playback of the image stream, further image stream or audio signal based on the speech recognition, e.g. based on the detected keywords and/or key phrases.

A third option is the control of the user interface via gaze detection. For example, the head-mounted display system may comprise a gaze-detection system 180, configured to determine a direction of a gaze of the user of the head-mounted display system. For example, the gaze-detection system may comprise one or more inward-facing cameras and/or one or more sensors for measuring the electro-magnetic field generated by the contraction of the muscles of the eye or eyes (of the user). In gaze detection, the gaze of the user is tracked. The detected gaze is correlated with a filed of view of the user, to determine which region of the field of view the user gazes at. In the field of view, the user interface is shown, with one or more virtual “buttons” (i.e. regions within the user interface), which can be actuated by gazing at them. For example, the control system may be configured to generate the display signal with the user interface with a user interface element (i.e. a “virtual button”) related to the control of the recording of the image stream and/or the playback of the recorded image stream. The control system may be configured to control the recording of the image stream and/or a playback of the recorded image stream based on the direction of the gaze of the user if the direction of the gaze of the user is directed towards the user interface element. For example, if the gaze intersects with the user interface element, the recording of the image stream, further image stream (including the still image) or audio signal may be started or stopped, or the playback of the image stream, further image stream (including the still image) or audio signal may be started or stopped. In more general terms, the control system may be configured to control the recording of the image stream, further image stream or audio signal and/or a playback of the recorded image stream, further image stream or audition signal based on the direction of the gaze of the user.

As mentioned above, the head-mounted display system may further comprise a gyroscope, e.g. in order to perform head tracking. For example, the field of view of the image stream may be controlled via the gyroscope. In other words, the control system may be configured to perform head tracking using the gyroscope, and to control the external device in order to adjust a field of view of the image stream. In addition, the control system may be configured to use the gyroscope to control the user interface.

The one or more interfaces 112 may correspond to one or more inputs and/or outputs for receiving and/or transmitting information, which may be in digital (bit) values according to a specified code, within a module, between modules or between modules of different entities. For example, the one or more interfaces 112 may comprise interface circuitry configured to receive and/or transmit information. In embodiments the one or more processors 114 may be implemented using one or more processing units, one or more processing devices, any means for processing, such as a processor, a computer or a programmable hardware component being operable with accordingly adapted software. In other words, the described function of the one or more processors 114 may as well be implemented in software, which is then executed on one or more programmable hardware components. Such hardware components may comprise a general-purpose processor, a Digital Signal Processor (DSP), a micro-controller, etc.

More details and aspects of the head-mounted display system and of the corresponding control system, method and computer program are mentioned in connection with the proposed concept or one or more examples described above or below (e.g. FIGS. 2 to 15). The head-mounted display system and of the corresponding control system, method and computer program may comprise one or more additional optional features corresponding to one or more aspects of the proposed concept or one or more examples described above or below.

FIG. 2 shows a schematic diagram of an example of a surgical microscope system 200 comprising a microscope 210 and a head-mounted display system 100, e.g. the head-mounted display system introduced in connection with FIG. 1a and/or 1b. As mentioned in connection with FIGS. 1a and/or 1b, the image stream received by the head-mounted display system may be received from an optical imaging sensor of a microscope of a surgical microscope system, e.g. from an optical imaging sensor of the microscope 210 of the surgical microscope system 200 shown in FIG. 2.

In general, a microscope is an optical instrument that is suitable for examining objects that are too small to be examined by the human eye (alone). For example, a microscope may provide an optical magnification of a sample. In modern microscopes, the optical magnification is often provided for a camera or an imaging sensor, such as an optical imaging sensor of the microscope 210 that is shown in FIG. 2. The microscope 210 may further comprise one or more optical magnification components that are used to magnify a view on the sample, such as an objective (i.e. lens). Although embodiments are described in connection with a microscope, they may also be applied, in a more general manner, to any optical device.

There are a variety of different types of microscopes. If the microscope is used in the medical or biological fields, the object being viewed through the microscope may be a sample of organic tissue, e.g. arranged within a petri dish or present in a part of a body of a patient. In the context of the present disclosure, the microscope 210 is part of a (neuro) surgical microscope system 200, e.g. a microscope to be used during a (neuro) surgical procedure. Such a system is shown in FIG. 2, for example. Accordingly, an object being viewed through the microscope, and shown in the imaging sensor data, may be a sample of organic tissue of a patient.

The surgical microscope system 100 shown in FIG. 1b comprises a number of optional components, such as a base unit with a (rolling) stand, a display, a (robotic or manual) arm which holds the microscope in place, and which is coupled to the base unit and to the microscope, and steering handles that are attached to the microscope. In the context of this application, the term “surgical microscope system” is used, in order to cover the portions of the system that are not part of the actual microscope (which comprises optical components), but which are used in conjunction with the microscope. For example, the head-mounted display system may be considered to be a part of the surgical microscope system.

The surgical microscope system is configured to provide an image stream to the head-mounted display system, e.g. based on imaging sensor data of an optical imaging sensor of the microscope 210 of the surgical microscope system 200. Therefore, the surgical microscope system may be configured to obtain the imaging sensor data from the optical imaging sensor of the microscope, to process the imaging sensor data and to provide the processed imaging sensor data to the head-mounted display system as image stream. For example, the optical imaging sensor may comprise or be an APS (Active Pixel Sensor)- or a CCD (Charge-Coupled-Device)-based imaging sensor. For example, in APS-based imaging sensors, light is recorded at each pixel using a photo-detector and an active amplifier of the pixel. APS-based imaging sensors are often based on CMOS (Complementary Metal-Oxide-Semiconductor) or S-CMOS (Scientific CMOS) technology. In CCD-based imaging sensors, incoming photons are converted into electron charges at a semiconductor-oxide interface, which are subsequently moved between capacitive bins in the imaging sensors by a control circuitry of the imaging sensors to perform the imaging. In various examples, the optical imaging sensor may comprise two or more individual sensors, e.g. two or more sensors for performing stereoscopic imaging, and/or two or more sensors for recording light in different wavelength bands.

In some examples, a field of view of the optical imaging sensor is controlled via head-tracking at the head-mounted display system. Consequently, the surgical microscope system may be configured to control the microscope, the optical imaging sensor, and/or a robotic arm based on the desired field of view, e.g. controlled by the head-mounted display system.

In general, the surgical microscope system may be configured to provide the image stream, and/or to receive control instructions via a wireline connection or via a wireless connection e.g. via Bluetooth or WiFi.

In general, the surgical microscope system may comprise a control system, with one or more interfaces, one or more processors and one or more storage devices, suitable for controlling the functionality of the surgical microscope system. For example, the control system may be configured to communicate with the head-mounted display system, e.g. to provide the image stream (and process the imaging sensor data), and/or to exchange control instructions.

More details and aspects of the surgical microscope system, the head-mounted display system and of the corresponding control system, method and computer program are mentioned in connection with the proposed concept or one or more examples described above or below (e.g. FIGS. 1a to 1b, 3 to 15). The surgical microscope system, the head-mounted display system and of the corresponding control system, method and computer program may comprise one or more additional optional features corresponding to one or more aspects of the proposed concept or one or more examples described above or below.

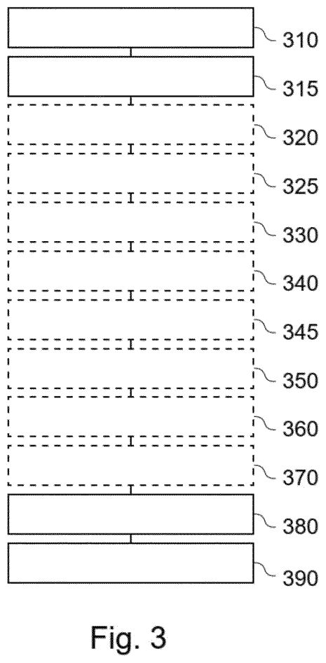

FIG. 3 shows a flow chart of an example of a method for a head-mounted display system. For example, the method may be performed (entirely) by the head-mounted display system. The method comprises receiving 310 an image stream from a device that is external to the head-mounted display system. The method comprises recording 315 the image stream using one or more storage devices of the head-mounted display system upon request of a user of the head-mounted display system. The method comprises generating 380 a display signal based on the image stream. The method comprises providing 390 the display signal to one or more displays of the head-mounted display system.

Optionally, the method may comprise one or more further features, e.g. one or more features introduced with the head-mounted display system introduced in connection with FIGS. 1a and/or 1b. For example, the method may comprise receiving 320 an audio signal. The method may comprise recording 325 the audio signal, and/or performing 330 speech recognition on the audio signal. For example, the method may comprise receiving 340 a further image stream, and recording 345 the further image stream. For example, the method may comprise extracting and/or recording 350 a still image, e.g. from the image stream or from the further image stream. For example, the method may comprise obtaining a control signal 360, e.g. from an electro-mechanical control element. For example, the method may comprise performing gaze tracking. The method may comprise controlling 370 the recording or playback, e.g. based on the control signal, based on the speech recognition, and/or based on the gaze tracking.

More details and aspects of the head-mounted display system and of the corresponding control system, method and computer program are mentioned in connection with the proposed concept or one or more examples described above or below (e.g. FIG. 1a to 2, 4 to 15). The head-mounted display system and of the corresponding control system, method and computer program may comprise one or more additional optional features corresponding to one or more aspects of the proposed concept or one or more examples described above or below.

Various aspects of the present disclosure relate to an apparatus for recording and replaying videos, audios and pictures directly on a head mounted visualization device (in the following also denoted “VRX viewer”), e.g. the head-mounted display system introduced in connection with FIGS. 1a to 3. The VRX-viewer may be a virtual reality enhanced head mounted viewer to record in real-time what the surgeon sees in the digital viewer during surgeries, in forms of videos and pictures, and also audio.

For example, the enhanced digital viewer (VRX-Viewer) may be equipped with additional multimedia features for at least one of recording, playing back/replaying, navigating through recorded files, receiving/sending video streams via Bluetooth or WiFi, processing voice commands and adding audio files to recordings, replaying sound or voice recordings during playback mode, voice recording, pictures recording, providing an OSD (on screen display/menu), and providing a status indication via an LED indicator etc.

The viewer is equipped with a storage device, such as a Micro-Secure Digital (SD) card and slot, enabling live recording during surgeries. In general, the proposed concept is based on a fully equipped viewer but is not limited to that. Hardware elements which cannot be integrated to the viewer based on restrictions for weight, size or ergonomics may lead to a peripherical storage option. Control and access to the processing unit may then be given via cable or wireless transmission.

The different features may be controlled by a multifunctional rotary knob or similar control and navigate mechanism. For example, this knob may have “push” and “rotate” functionality. For example, a rotation of the rotary know may be used to navigate through the OSD (on screen display/menu) and a push may be used to confirm the selected feature/mode.

In another example the features may be controlled with head tracking (use of gyroscopes), or gaze/eye-tracking (looking at virtual buttons displayed in the screens in the viewer), or voice (using the microphone with specific words). The Viewer may also be equipped with buttons for volume control, ON/OFF function, record, playback, etc. Additional software elements can be added and assigned to these buttons. The software may enable full recording functionality, playback functionality and other features.

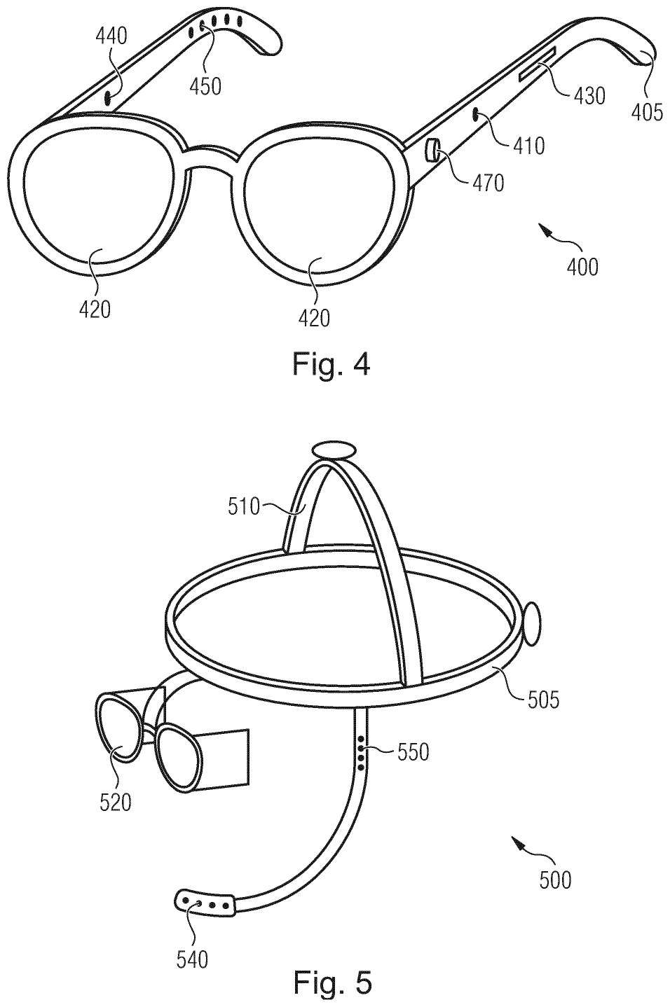

FIG. 4 shows a schematic diagram of an example of a head-mounted display system 400. The head-mounted display system is substantially shaped liked glasses. The head-mounted display system 400 comprises displays 420, which are integrated within a frame 405 of the glasses. The frame, or holding structure, holds the other elements, such as status Light-Emitting Diode 410, the displays 420, a Micro-SD card slog 430, a microphone 440, a speaker or speakers 450, and a controlling knob 470.

FIG. 5 shows a schematic diagram of another example of a head-mounted display system 500. The head-mounted display system of FIG. 5 comprises a round holding structure 505, which is coupled to a headband 510 and can be placed on the head of the wearer. The head-mounted display system 500 comprises viewer channels 520 (i.e. ocular displays), a microphone 540 and a speaker 550, the microphone 540 and speaker 550 being integrated within a microphone arm that is part of the holding structure.

For example, the head-mounted display systems may comprise one or more of a gyro sensor, a microphone 440/540, a sound speaker 450/550, an LED (no power/status indication) 410, Bluetooth/WiFi (Connection for Streaming), a rotary knob 470 to activate OSD. For example, the head-mounted display systems may comprise a menu with multiple layers for the user to record, replay, toggle between records, and/or change the speed of playback etc. For example, the multi-layer menu may also be used to setup the device, e.g. to format the SD card.

In the following, a number of flow charts are presented of examples of methods related to video recording, video replaying, and voice recording. For example, the methods may be performed with the help of the head-mounted display systems introduced in connection with FIGS. 1a to 5. ‘In the following, an “SD card” is used representatively for the one or more storage devices. However, other types of storage devices may be used instead of the SD card.

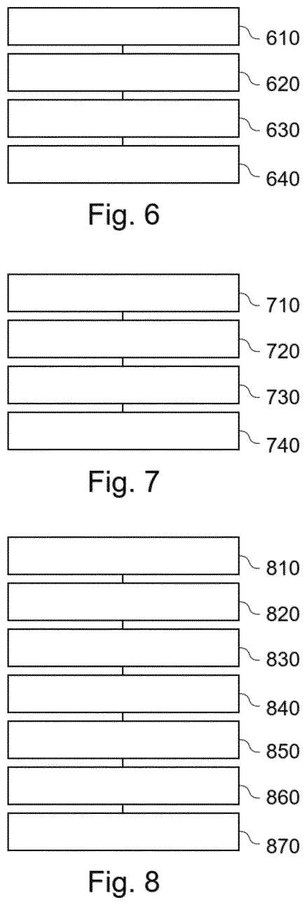

FIG. 6 shows a flow chart of a first example related to video recording, e.g. of a first method for video recording. In the method, the user presses a dedicated button 610 so the video starts being saved in an SD card 620. If the user wants to terminate the recording, the user presses the dedicated button 630 and the full video is stored in the SD card 640.

FIG. 7 shows a flow chart of a second example related to video recording, e.g. of a second method for video recording. In the method, the user says “video recording start” 710 so the video starts being saved in an SD card 720. If the user wants to terminate the recording, the user says “video recording stop” 730 and the full video is stored in the SD card 740.

FIG. 8 shows a flow chart of a third example related to video recording, e.g. of a third method for video recording. In the method, the user presses a dedicated button 810 so a menu is displayed on the viewer's screens 820. The user then turns the dedicated button to select the video recording function 830 and presses the dedicated button to confirm the choice 840. Consequently, the video starts being saved in the SD card 850. If the user wants to terminate the recording, the user presses the dedicated button 860, and the full video is stored in the SD card 870.

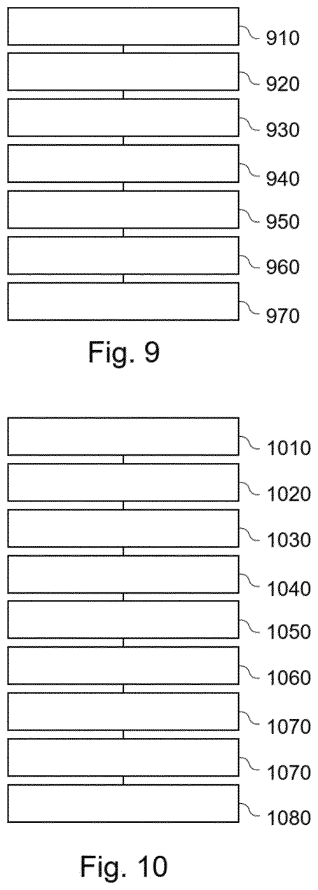

FIG. 9 shows a flow chart of a fourth example related to video recording, e.g. of a fourth method for video recording. In the method, the user looks at the virtual menu button displayed on the viewer's screens 910 so the menu is displayed on the viewer's screens 920. The user looks at the virtual video recording button on the viewer's screens 930. Subsequently, the virtual button “stop recording” is being displayed on the viewer's screens 940 and the video starts being saved in SD card 950. If the user wants to terminate the recording, the user looks at the virtual “stop recording” button displayed on the viewer's screens 960 and the full video stored in the SD card 970.

FIG. 10 shows a flow chart of a first example related to video replaying, e.g. of a first method for video replaying. In the method, the user presses dedicated button 1010 so the menu is displayed on the viewer's screens 1020. Then the user turns the dedicated button to select the video replay function 1030 and presses the dedicated button to confirm the choice 1040. Subsequently, a menu with the list of recorded video displayed on the viewer's screens 1050. The user turns the dedicated button to select the video to be replayed in the viewer's screens 1060 and presses the dedicated button to confirm the choice 1070. Accordingly, the selected video is being played in the viewer's screens 1080.

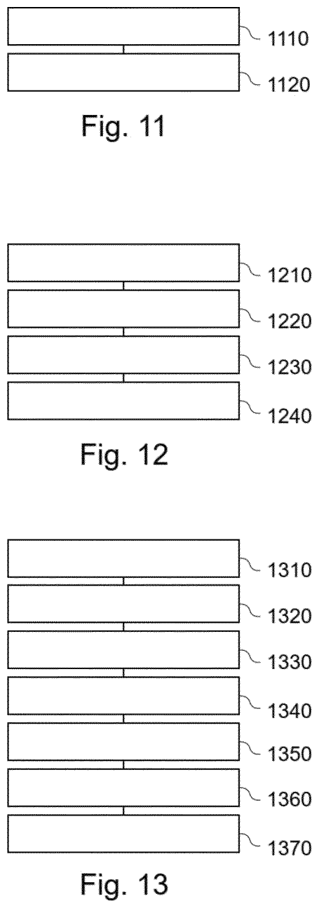

FIG. 11 shows a flow chart of a second example related to video replaying, e.g. of a second method for video replaying. In the method, the user says “replay video n” 1110, so video n is being played in the viewer's screens 1120.

FIG. 12 shows a flow chart of a first example related to voice recording, e.g. of a first method for voice recording. In the method, the user says “voice recording start” 1210 so the voice starts being saved in the SD card 1220. When the user says “voice recording stop” 1230, the full speech is stored in the SD card 1240.

FIG. 13 shows a flow chart of a second example related to voice recording, e.g. of a second method for voice recording. In the method, the user presses a dedicated button 1310 so the menu is displayed on the viewer's screens 1320. The user turns the dedicated button to select the voice recording function 1330 and presses the dedicated button to confirm the choice 1340, so the voice starts being saved in the SD card 1350. When the user presses dedicated button 1360 (again), the full speech is stored in the SD card 1370.

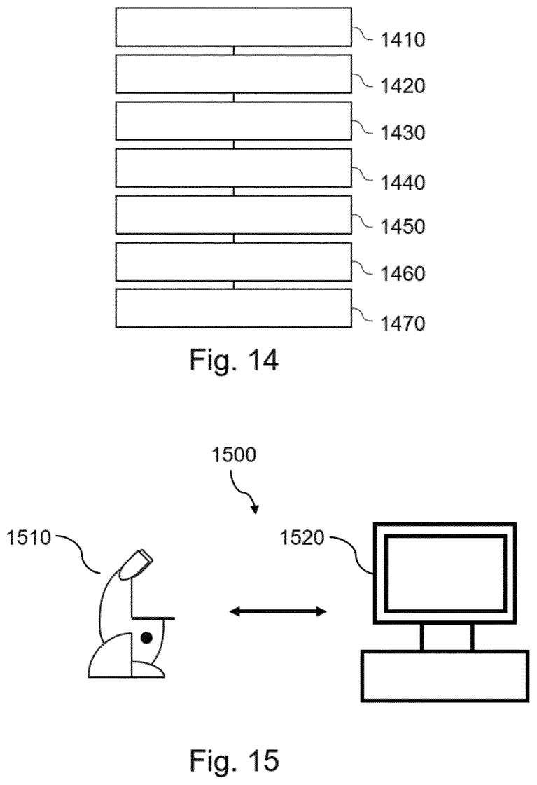

FIG. 14 shows a flow chart of a third example related to voice recording, e.g. of a third method for voice recording. In the method, the user looks at the virtual menu button displayed on the viewer's screen 1410 so the menu is displayed on the viewer's screens 1420. The user then looks at the virtual voice recording button displayed on the viewer's screens 1430, so the virtual button “stop recording” is displayed on the viewer's screens 1440 and the voice starts being saved in SD card 1450. When the user looks at the virtual “stop” recording button displayed on the viewer's screens 1460 the full speech stored in SD card 1470.

As used herein the term “and/or” includes any and all combinations of one or more of the associated listed items and may be abbreviated as “/”.

Although some aspects have been described in the context of an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus.

Some embodiments relate to a microscope comprising a system as described in connection with one or more of the FIGS. 1 to 14. Alternatively, a microscope may be part of or connected to a system as described in connection with one or more of the FIGS. 1 to 14. FIG. 15 shows a schematic illustration of a system 1500 configured to perform a method described herein. The system 1500 comprises a microscope 1510 and a computer system 1520. The microscope 1510 is configured to take images and is connected to the computer system 1520. The computer system 1520 is configured to execute at least a part of a method described herein. The computer system 1520 may be configured to execute a machine learning algorithm. The computer system 1520 and microscope 1510 may be separate entities but can also be integrated together in one common housing. The computer system 1520 may be part of a central processing system of the microscope 1510 and/or the computer system 1520 may be part of a subcomponent of the microscope 1510, such as a sensor, an actor, a camera or an illumination unit, etc. of the microscope 1510.

The computer system 1520 may be a local computer device (e.g. personal computer, laptop, tablet computer or mobile phone) with one or more processors and one or more storage devices or may be a distributed computer system (e.g. a cloud computing system with one or more processors and one or more storage devices distributed at various locations, for example, at a local client and/or one or more remote server farms and/or data centers). The computer system 1520 may comprise any circuit or combination of circuits. In one embodiment, the computer system 1520 may include one or more processors which can be of any type. As used herein, processor may mean any type of computational circuit, such as but not limited to a microprocessor, a microcontroller, a complex instruction set computing (CISC) microprocessor, a reduced instruction set computing (RISC) microprocessor, a very long instruction word (VLIW) microprocessor, a graphics processor, a digital signal processor (DSP), multiple core processor, a field programmable gate array (FPGA), for example, of a microscope or a microscope component (e.g. camera) or any other type of processor or processing circuit. Other types of circuits that may be included in the computer system 1520 may be a custom circuit, an application-specific integrated circuit (ASIC), or the like, such as, for example, one or more circuits (such as a communication circuit) for use in wireless devices like mobile telephones, tablet computers, laptop computers, two-way radios, and similar electronic systems. The computer system 1520 may include one or more storage devices, which may include one or more memory elements suitable to the particular application, such as a main memory in the form of random-access memory (RAM), one or more hard drives, and/or one or more drives that handle removable media such as compact disks (CD), flash memory cards, digital video disk (DVD), and the like. The computer system 1520 may also include a display device, one or more speakers, and a keyboard and/or controller, which can include a mouse, trackball, touch screen, voice-recognition device, or any other device that permits a system user to input information into and receive information from the computer system 1520.

Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

Some embodiments according to the invention comprise a data carrier having electronically readable control signals, which are capable of cooperating with a programmable computer system, such that one of the methods described herein is performed.

Generally, embodiments of the present invention can be implemented as a computer program product with a program code, the program code being operative for performing one of the methods when the computer program product runs on a computer. The program code may, for example, be stored on a machine-readable carrier.

Other embodiments comprise the computer program for performing one of the methods described herein, stored on a machine-readable carrier.

In other words, an embodiment of the present invention is, therefore, a computer program having a program code for performing one of the methods described herein, when the computer program runs on a computer.

A further embodiment of the present invention is, therefore, a storage medium (or a data carrier, or a computer-readable medium) comprising, stored thereon, the computer program for performing one of the methods described herein when it is performed by a processor. The data carrier, the digital storage medium or the recorded medium are typically tangible and/or non-transitionary. A further embodiment of the present invention is an apparatus as described herein comprising a processor and the storage medium.

A further embodiment of the invention is, therefore, a data stream or a sequence of signals representing the computer program for performing one of the methods described herein. The data stream or the sequence of signals may, for example, be configured to be transferred via a data communication connection, for example, via the internet.

A further embodiment comprises a processing means, for example, a computer or a programmable logic device, configured to, or adapted to, perform one of the methods described herein.

A further embodiment comprises a computer having installed thereon the computer program for performing one of the methods described herein.

A further embodiment according to the invention comprises an apparatus or a system configured to transfer (for example, electronically or optically) a computer program for performing one of the methods described herein to a receiver. The receiver may, for example, be a computer, a mobile device, a memory device or the like. The apparatus or system may, for example, comprise a file server for transferring the computer program to the receiver.

In some embodiments, a programmable logic device (for example, a field programmable gate array) may be used to perform some or all of the functionalities of the methods described herein. In some embodiments, a field programmable gate array may cooperate with a microprocessor in order to perform one of the methods described herein. Generally, the methods are preferably performed by any hardware apparatus.

### LIST OF REFERENCE SIGNS

100 Head-mounted display system
105 Holding structure, frames
110 System
112 One or more interfaces
114 One or more processors
120 Display
130 Storage device
140 Microphone
150 Speaker
160 Camera
170 Electro-mechanical control element, rotary button
180 Gaze-detection system
200 Surgical microscope system
205 Stand
210 Microscope
220 System
230 Display
240 Arm
310 Receiving an image stream
315 Recording the image stream
320 Receiving an audio signal
325 Recording the audio signal
330 Performing speech recognition
340 Receiving a further image stream
345 Recording the further image stream
350 Extracting/recording a still image
360 Obtaining a control signal
370 Controlling the recording or playback
380 Generating a display signal
390 Providing the display signal to one or more displays
400 Head-mounted display system
405 Frame
410 Status LED
420 Displays
430 Micro SD Slot
440 Microphone
450 Speaker
470 Controlling Knob
500 Head-mounted display system
505 Holding structure
510 Headband
520 Viewer Channels
540 Microphone
550 Speaker
610 User presses dedicated button
620 Video starts being saved in SD card
630 User presses dedicated button
640 Full video stored in SD card
710 User says “video recording start”
720 Video starts being saved in SD card
730 User says “video recording stop”
740 Full video stored in SD card
810 User presses dedicated button
820 Menu displayed on the viewer's screens
830 User turns the dedicated button to select the video recording function
840 User presses dedicated button to confirm the choice
850 Video starts being saved in SD card
860 User presses dedicated button
870 Full video stored in SD card
910 User looks at the virtual menu button displayed on the viewer's screens
920 Menu displayed on the viewer's screens
930 User looks at the virtual video recording button on the viewer's screens
940 The virtual button “stop recording” is being displayed on the viewer's screens
950 Video starts being saved in SD card
960 User looks at the virtual “stop recording” button displayed on the viewer's screens
970 Full video stored in SD card
1010 User presses dedicated button
1020 Menu displayed on the viewer's screens
1030 User turns the dedicated button to select the video replay function
1040 User presses dedicated button to confirm the choice
1050 Menu with the list of recorded video displayed on the viewer's screens
1060 User turns the dedicated button to select the video to be replayed in the viewer's screens
1070 User presses dedicated button to confirm the choice
1080 Selected video is being played in the viewer's screens
1110 User says “replay video n”
1120 Video n is being played in the viewer's screens
1210 User says “voice recording start”
1220 Voice starts being saved in SD card
1230 User says “voice recording stop”
1240 Full speech is stored in SD card
1310 User presses dedicated button
1320 Menu displayed on the viewer's screens
1330 User turns the dedicated button to select the voice recording function
1340 User presses dedicated button to confirm the choice
1350 Voice recording starts being saved in SD card
1360 User presses dedicated button
1370 Full speech is stored in SD card
1410 The user looks at the virtual menu button displayed on the viewer's screen
1420 Menu displayed on the viewer's screens
1430 User looks at the virtual voice recording button displayed on the viewer's screens
1440 Virtual button “stop recording” is displayed on the viewer's screens
1450 Voice starts being saved in SD card
1460 User looks at the virtual “stop” recording button displayed on the viewer's screens
1470 Full speech is stored in SD card
1500 System
1510 Microscope
1520 Computer system

1. A head-mounted display system comprising:
one or more displays;
one or more storage devices; and
a control system comprising one or more interfaces and one or more processors, wherein the control system is configured to:
receive an image stream from an external device,
record the image stream using the one or more storage devices upon request of a user of the head-mounted display system,
generate a display signal based on the image stream, and
provide the display signal to the one or more displays.

2. The head-mounted display system according to claim 1, wherein the control system is configured to generate the display signal alternately based on a currently received version of the image stream or based on a recorded version of the image stream.

3. The head-mounted display system according to claim 1, wherein the control system is configured to extract a still image from the image stream upon request of the user of the head-mounted display system, and to record the still image using the one or more storage devices.

4. The head-mounted display system according to claim 1, wherein the head-mounted display system comprises at least one microphone, wherein the control system is configured to receive an audio signal from the at least one microphone, and to record the audio signal using the one or more storage devices upon request of the user of the head-mounted display system.

5. The head-mounted display system according to claim 1, wherein the head-mounted display system comprises at least one camera, wherein the control system is configured to receive a further image stream from the at least one camera, and to record the further image stream using the one or more storage devices upon request of a user of the head-mounted display system.

6. The head-mounted display system according to claim 5, wherein the control system is configured to generate the display signal alternately based on the image stream or based on the further image stream.

7. The head-mounted display system according to claim 1, comprising an electro-mechanical control element that is coupled to the control system, wherein the control system is configured to obtain a control signal from the electro-mechanical control element, and to control the recording of the image stream and/or a playback of the recorded image stream based on the control signal.

8. The head-mounted display system according to claim 7, wherein the electro-mechanical control element is a rotary button, configured to generate the control signal based on a rotation and actuation of the rotary button.

9. The head-mounted display system according to claim 1, wherein the head-mounted display system comprises at least one microphone, wherein the control system is configured to receive an audio signal from the at least one microphone, to perform speech recognition on the audio signal, and to control the recording of the image stream and/or a playback of the recorded image stream based on the speech recognition.

10. The head-mounted display system according to claim 1, comprising a gaze-detection system, configured to determine a direction of a gaze of the user of the head-mounted display system, wherein the control system is configured to control the recording of the image stream and/or a playback of the recorded image stream based on the direction of the gaze of the user.

11. The head-mounted display system according to claim 1, comprising a holding structure that is mechanically coupled to the one or more displays, the one or more storage devices and the control system.

12. The head-mounted display system according to claim 1, wherein the control system is configured to receive the image stream from a microscope of a surgical microscope system.

13. A surgical microscope system comprising a microscope and the head-mounted display system according to claim 12.

14. A method for a head-mounted display system, the method being performed by the head-mounted display system, the method comprising:
receiving an image stream from a device that is external to the head-mounted display system;
recording the image stream using one or more storage devices of the head-mounted display system upon request of a user of the head-mounted display system;
generating a display signal based on the image stream; and
providing the display signal to one or more displays of the head-mounted display system.

15. A non-transitory, computer-readable medium comprising a program code that, when the program code is executed on a processor, a computer, or a programmable hardware component, causes the processor, computer, or programmable hardware component to perform the method of claim 14.
